(19) United States
(12) Patent Application Publication
(10) Pub. No.: US 2024/0082540 A1
(43) Pub. Date: Mar. 14, 2024

(54) CATHETERS HAVING MULTIPLE COIL LAYERS, AND ASSOCIATED SYSTEMS AND METHODS

(71) Applicant: Inari Medical, Inc., Irvine, CA (US)

(72) Inventors: Thomas Robert Brodt, Long Beach, CA (US); John Coleman Thress, Capistrano Beach, CA (US); Benjamin Edward Merritt, San Clemente, CA (US); Miguel Rendon Flores, Fallbrook, CA (US); Donald Joseph Fuller, San Clemente, CA (US)

(21) Appl. No.: 18/463,960

(22) Filed: Sep. 8, 2023

Related U.S. Application Data
(60) Provisional application No. 63/405,251, filed on Sep. 9, 2022.

(51) Int. Cl. A61M 25/00; H01F 27/28

(52) U.S. Cl. CPC A61M 25/005; A61M 25/0012; H01F 27/2823

(57) ABSTRACT

Disclosed herein are catheters having multiple coil layers, and associated systems and methods. In some embodiments, a catheter includes an inner liner, a plurality of wires over the inner liner, and an outer sheath over the wires. The wires are coiled about a longitudinal axis of the catheter in a first direction to form a first coil layer over the inner liner, and the wires are coiled about the longitudinal axis in a second direction, opposite the first direction, over the first coil layer to form a second coil layer over the first coil layer. The wires each include a continuous/self-terminating end portion wherein the wire switches from the first direction to the second direction and transitions from the inner coil layer to the outer coil layer.

### CROSS-REFERENCE TO RELATED APPLICATION

This application claims the benefit of U.S. Provisional Patent Application No. 63/405,251, filed Sep. 9, 2022, and titled “CATHETERS HAVING MULTIPLE COIL LAYERS, AND ASSOCIATED SYSTEMS AND METHODS,” which is incorporated herein by reference in its entirety.

### TECHNICAL FIELD

The present technology generally relates to catheters having multiple coil layers and, more particularly, to catheters having adjacent coil layers formed from the same wires that are (i) coiled in a first direction to form an inner coil layer and (ii) coiled in a second direction opposite the first direction to form an outer coil layer over the inner coil layer.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. Infarction does not develop or is greatly limited if the flow of blood is reestablished rapidly. Failure to reestablish blood flow can lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT arises most commonly when there is a propensity for stagnated blood (e.g., long distance air travel, immobility, etc.) and clotting (e.g., cancer, recent surgery, such as orthopedic surgery, etc.). DVT causes harm by: (1) obstructing drainage of venous blood from the legs leading to swelling, ulcers, pain, and infection, and (2) serving as a reservoir for blood clots to travel to other parts of the body including the heart, lungs, brain (stroke), abdominal organs, and/or extremities.

In the pulmonary circulation, the undesirable material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. One common surgical technique, an embolectomy, involves incising a blood vessel and introducing a balloon-tipped device (such as the Fogarty catheter) to the location of the occlusion. The balloon is then inflated at a point beyond the clot and used to translate the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and the vessel will re-stenos after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage and in many patients the agents cannot be used at all.

Various devices exist for performing a thrombectomy or removing other foreign material. However, such devices have been found to have structures which are either highly complex, cause trauma to the treatment vessel, or lack sufficient retaining structure and thus cannot be appropriately fixed against the vessel to perform adequately. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system including a catheter in accordance with embodiments of the present technology.

FIG. 2 is a side cross-sectional view of the catheter of FIG. 1 taken along a line C-C in FIG. 1 in accordance with embodiments of the present technology.

FIGS. 3A and 3B are enlarged isometric views of portions of the catheter of FIG. 1 in accordance with embodiments of the present technology.

FIGS. 3C-3G are side views of inner and outer coil layers of the catheter of FIG. 1 at a distal portion of the catheter in accordance with embodiments of the present technology.

FIG. 4 is an enlarged isometric view of a portion of the catheter of FIG. 1 in accordance with additional embodiments of the present technology.

FIG. 5 is an enlarged isometric view of a portion of the catheter of FIG. 1 in accordance with additional embodiments of the present technology.

FIG. 6 is a side cross-sectional view of the catheter of FIG. 1 taken along the line C-C in FIG. 1 in accordance with additional embodiments of the present technology.

FIG. 7 is a side cross-sectional view of the catheter of FIG. 1 taken along the line C-C in FIG. 1 in accordance with additional embodiments of the present technology.

FIG. 8 is a side cross-sectional view of the catheter of FIG. 1 taken along the line C-C in FIG. 1 in accordance with additional embodiments of the present technology.

FIG. 9 is a flow diagram of a process or method for manufacturing the catheter of FIG. 1 in accordance with embodiments of the present technology.

FIGS. 10A and 10B are side views of a distal portion of the catheter of FIG. 1 during a procedure for removing clot material from within a blood vessel BV of a patient in accordance with embodiments of the present technology.

### DETAILED DESCRIPTION

The present technology is generally directed to catheters (e.g., aspiration catheters) having multiple coil layers, and associated systems and method. In some embodiments, a catheter configured in accordance with the present technology includes an inner liner, a plurality of wires over the inner liner, and an outer sheath over the wires. The wires are coiled about a longitudinal axis of the catheter in a first direction to form a first coil layer over the inner liner, and the wires are coiled about the longitudinal axis in a second direction, opposite the first direction, over the first coil layer to form a second coil layer over the first coil layer. The wires each include a continuous/self-terminating end portion where the wire switches from the first direction to the second direction and transitions from the inner coil layer to the outer coil layer.

In some aspects of the present technology the inner and outer coil layers can be configured to provide the catheter with select characteristics over the entire length of the catheter or in select regions of the catheter, such as a selected flexibility, pushability, torqueability, kink resistance, hoop strength, and/or other characteristic known in the art of catheters. For example, the number of wires used to form the inner and outer coil layers and/or the pitch between the wires can be varied.

In additional aspects of the present technology, the arrangement of the inner and outer coil layers can enable the catheter to be steered to and positioned in difficult-to-reach (e.g., tortuous) regions of the anatomy (e.g., venous anatomy) of a patient while still having a relatively large size (e.g., 20 French, 24 French, greater than 24 French). More particularly, the inner and outer coil layers can give the catheter a torqueability and pushability that is similar to conventional catheters including a braided structure (e.g., a braided mesh). Similarly, the inner and outer coil layers can provide a hoop strength and flexibility similar to or greater than conventional catheters including a single-filar coil structure while also reducing kinking around tight bend radii. Further, the inner and outer coil layers allow the catheter to have these characteristics without including a braided structure or other reinforcement structures, which allows the catheter to be manufactured with a relatively thinner inner liner and/or outer sheath and results in a catheter having a thinner wall. This can enable the inner diameter of the catheter to be larger than other catheters labeled at the same outer diameter French size. A larger inner diameter can be optimal for generating aspirational flow rate (e.g., increasing aspirational flow rate) during thrombectomy or embolectomy leading to more complete clot removal.

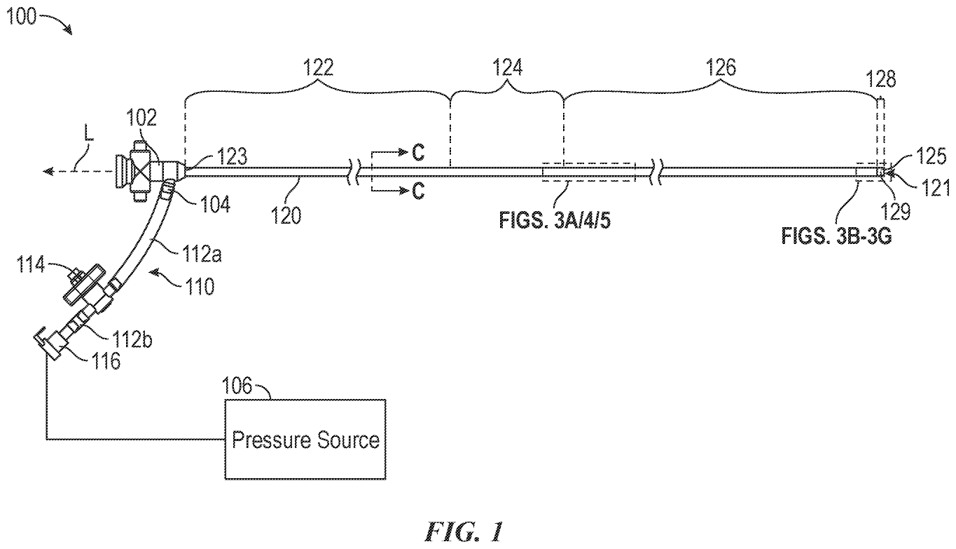

Certain details are set forth in the following description and in FIGS. 1-10B to provide a thorough understanding of various embodiments of the present technology. In other instances, well-known structures, materials, operations, and/or systems often associated with intravascular procedures, clot removal procedures, catheters, and the like are not shown or described in detail in the following disclosure to avoid unnecessarily obscuring the description of the various embodiments of the technology. Moreover, although reference is primarily made to aspiration catheters and catheters for use in clot removal procedures, the catheters of the present technology can be other types of catheters and/or can be used in other types of medical procedures. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

As used herein, unless expressly indicated otherwise, the terms “about,” “approximately,” “substantially” and the like mean within plus or minus 10% of the stated value. To the extent any materials incorporated herein by reference conflict with the present disclosure, the present disclosure controls. The headings provided herein are for convenience only and should not be construed as limiting the subject matter disclosed.

### I. SELECTED EMBODIMENTS OF CLOT TREATMENT SYSTEMS

FIG. 1 is a partially schematic side view of a clot treatment system 100 in accordance with embodiments of the present technology. The clot treatment system 100 can also be referred to as an aspiration assembly, a clot removal system, and/or a thrombectomy system. In the illustrated embodiment, the clot treatment system 100 includes a tubing assembly 110 fluidly coupled to a catheter 120 via a valve 102. In general, the clot treatment system 100 (i) can include features generally similar or identical to those of the clot treatment systems described in detail in U.S. patent application Ser. No. 16/636,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter 120 includes (i) a proximal region or portion 122, (ii) an intermediate region 124 adjacent to and distal of the proximal region 122, (iii) a distal region 126 adjacent to and distal of the intermediate region 124, and (iv) a distal tip region 128 adjacent to and distal of the distal region 126 (collectively “the regions 122, 124, 126, 128”). The catheter 120 further defines a lumen 121 extending entirely therethrough from the proximal region 122 to the distal tip region 128. The proximal region 122 defines a proximal terminus 123 of the catheter 120, and the distal tip region 128 defines a distal terminus 125 of the catheter 120. In the illustrated embodiment, the distal tip region 128 includes a marker band 129, such as a radiopaque marker configured to facilitate visualization of the position of the catheter 120 during a medical procedure (e.g., a clot removal procedure) using the catheter 120. In other embodiments, the marker band 129 can be omitted or positioned at a different location along the catheter 120, and/or the catheter 120 can include additional marker bands to facilitate visualization of the catheter 120.

In some embodiments, the proximal region 122 has a first length, the intermediate region 124 has a second length less than the first length, the distal region 126 has a third length greater than the second length but less than the first length, and the distal tip region 128 has a fourth length less than the first, second, and third lengths. For example, the first length can be between about 20.00-50.00 inches (e.g., about 22.0 inches), the second length can be between about 2.0-3.0 inches (e.g., about 2.50 inches), the third length can be between about 10.00-20.00 inches (e.g., about 16.00 inches), and the fourth length can be between about 0.10-0.50 inch (e.g., about 0.15 inch). In other embodiments, the lengths of one or more of the regions 122, 124, 126, 128 can be different. As used herein with reference to the first through fourth lengths, the term “about” means within plus or minus 0.50 inch of the stated length. In some embodiments, the catheter 120 can have varying flexibilities, shapes, thicknesses, and/or other properties in/along the various regions 122, 124, 126, 128, as described in greater detail below. The lengths of the regions 122, 124, 126, 128 relative to one another in the figures may not be drawn to scale.

The valve 102 is fluidly coupled to the lumen 121 of the catheter 120 and can be integral with or coupled to the proximal region 122 of the catheter 120. In some embodiments, the valve 102 is a hemostasis valve that is configured to maintain hemostasis during a clot removal procedure by inhibiting or preventing fluid flow in the proximal direction through the valve 102 as various components such as delivery sheaths, pull members, guidewires, interventional devices, other aspiration catheters, and so on are inserted through the valve 102 to be delivered through the catheter 120 to a treatment site in a blood vessel. The valve 102 includes a branch or side port 104 configured to fluidly couple the lumen 121 of the catheter 120 to the tubing assembly 110. In some embodiments, the valve 102 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 106, such as a syringe. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 106 and/or other suitable components. In some embodiments, the fluid control device 114 is a stopcock that is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 121 of the catheter 120 to the pressure source 106. In some embodiments, the connector 116 is a quick-release connector (e.g., a quick disconnect fitting) that enables rapid coupling/decoupling of the catheter 120 and the fluid control device 114 to/from the pressure source 106.

### II. SELECTED EMBODIMENTS OF CATHETERS HAVING SELF-TERMINATING COILS

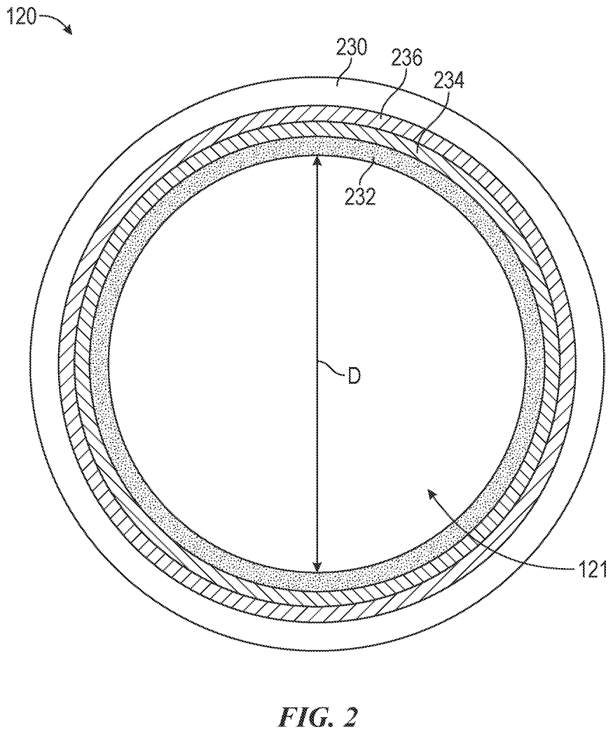

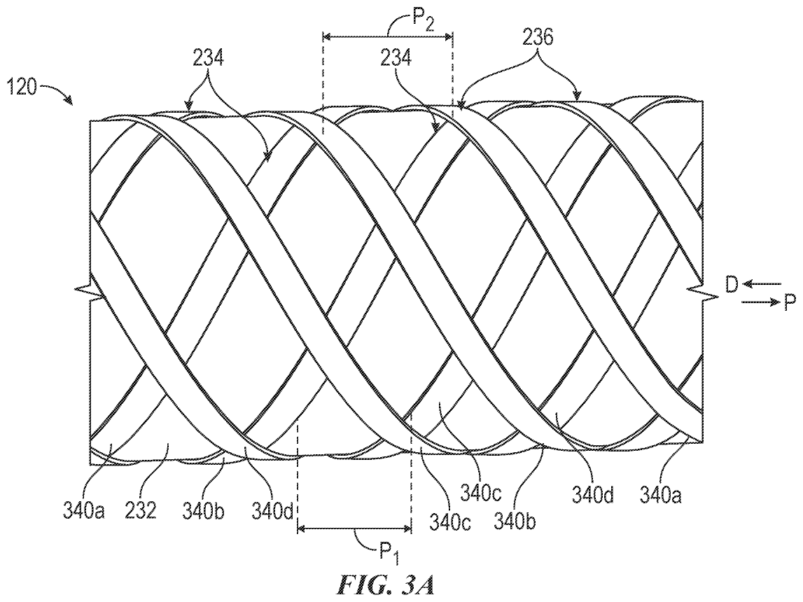

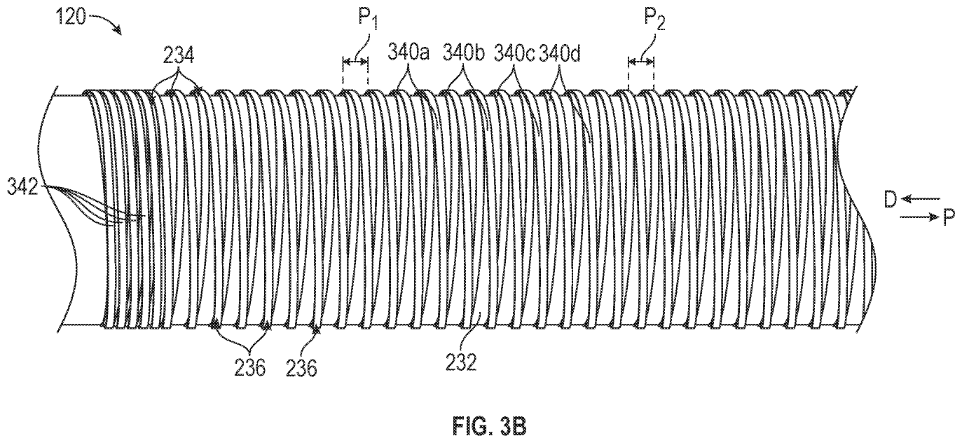

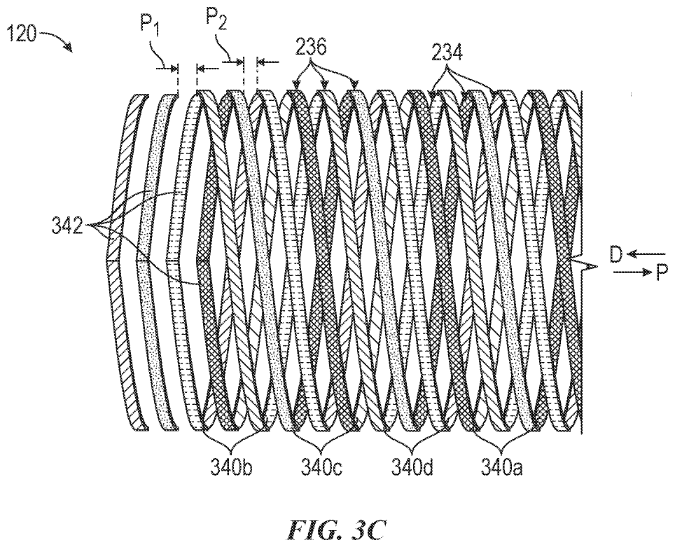

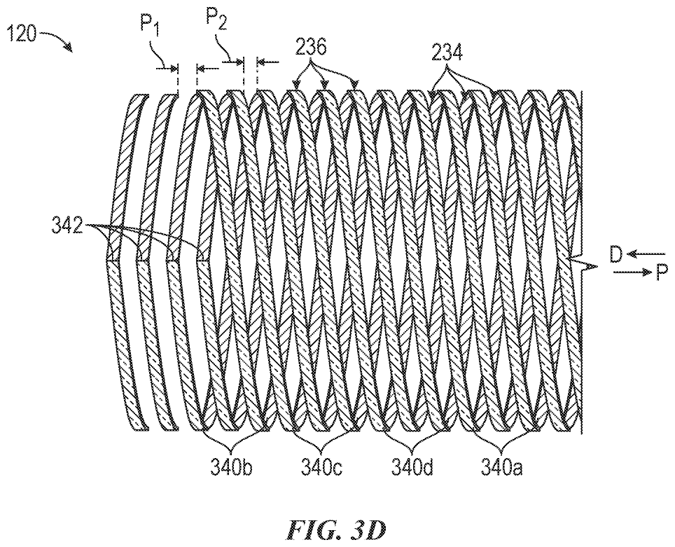

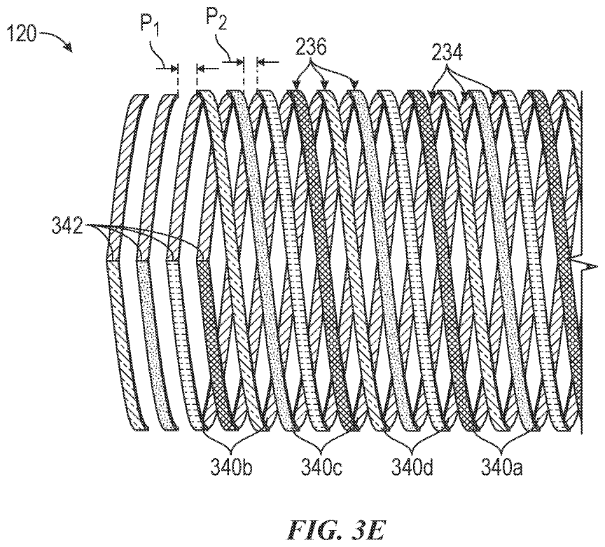

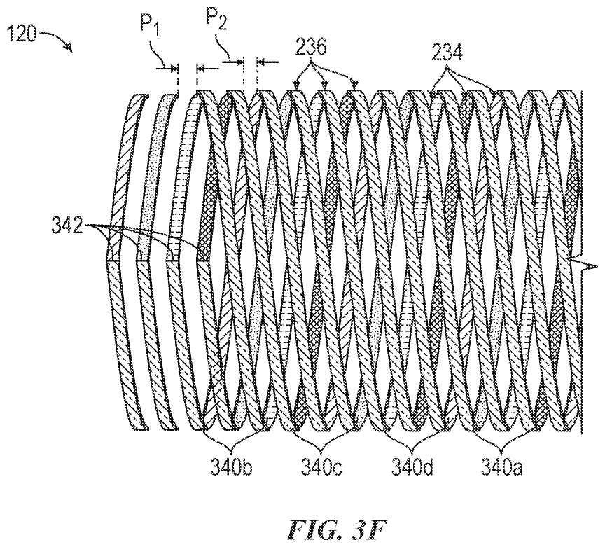

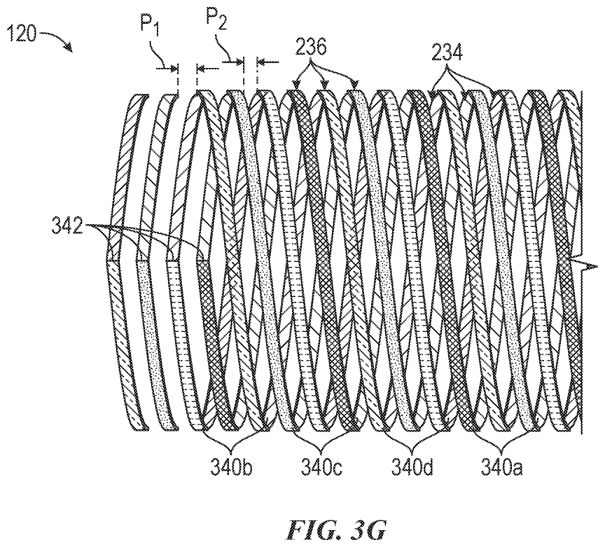

FIG. 2 is a side cross-sectional view of the catheter 120 taken along the line C-C in FIG. 1 in accordance with embodiments of the present technology. In the illustrated embodiment, the catheter 120 includes an outer sheath 230 and an inner liner 232 extending through/defining each of the regions 122, 124, 126, 128. The outer sheath 230 is positioned over (e.g., radially outside of) the inner liner 232. The outer sheath 230 can also be referred to as an outer jacket, an outer shaft, an outer layer, or the like, and the inner liner 232 can also be referred to as an inner layer, an inner sheath, an inner shaft, or the like. In the illustrated embodiment, the catheter 120 further includes an inner coil layer 234 (e.g., a first coil layer) and an outer coil layer 236 (e.g., a second coil layer) extending over/about the inner coil layer 234. As described in greater detail below with reference to FIGS. 3A-3G, the inner coil layer 234 and the outer coil layer 236 (collectively “the coil layers 234, 236”) can comprise a plurality of individual wires that are wound around the inner liner 232. The coil layers 234, 236 can extend along an entire length of the catheter 120 through each of the regions 122, 124, 126, 128, or can extend only partially along the length of catheter 120 (e.g., in the distal region 126 and the distal tip region 128). The coil layers 234, 236 can be referred to collectively as a reinforcement structure or the like.

The outer sheath 230 can be formed from a plastic material, elastomeric material, and/or thermoplastic elastomer (TPE) material. In some embodiments, the outer sheath 230 can be formed from a TPE manufactured by Arkema S.A., of Colombes, France, such as the TPEs manufactured under the trademark “Pebax.” In some embodiments, the outer sheath 230 can have a varying hardness (e.g., durometer), thickness, flexibility, rigidity, and/or other property in one or more of the different regions 122, 124, 126, 128. For example, the outer sheath 230 can have a first hardness along the proximal region 122, a second hardness along the intermediate region 124 that is less than the first hardness, a third hardness along the distal region 126 that is less than the first hardness and the second hardness, and a fourth hardness in the distal tip region 128 that is greater than third hardness. In some embodiments, the first hardness and the fourth hardness can each be between about 65D-75D (e.g., about 72D), the second hardness can be between about 45D-60D (e.g., about 45D, about 50D), and the third hardness can be between about 25D-40D (e.g., about 25D, about 30D, about 35D). As used herein with reference to the first through fourth hardnesses, the term “about” means within plus or minus 2D of the stated hardness. In other embodiments, one or more of the regions 122, 124, 126, 128 can have a different hardness.

The inner liner 232 defines the lumen 121 and, in some embodiments, can be formed of a lubricious material that facilitates the movement (e.g., distal advancement, proximal retraction) of various components through the lumen 121, such as delivery sheaths, pull members, guidewires, interventional devices, other aspiration catheters, and the like. In some embodiments, the inner liner 232 can be formed from a polymer material, a fluoropolymer material (e.g., polytetrafluoroethylene (PTFE)), and/or another material having a high degree of lubricity. The inner liner 232 defines an inner diameter D of the catheter 120. The thicknesses of the outer sheath 230, the inner liner 232, and the coil layers 234, 236 relative to one another and/or to the inner diameter D may not be to scale in FIG. 2. For example, one of ordinary skill in the art will understand that the thicknesses of these components are shown for clarity but that the catheter 120 can have a relatively thin wall (e.g., comprising the outer sheath 230, the inner liner 232, and the coil layers 234, 236) compared to the inner diameter D. In some embodiments, the inner diameter D is greater than about 6 French, greater than about 10 French, greater than about 16 French, greater than about 20 French, greater than about 24 French, or greater. In some embodiments, the inner diameter D is about 8 French, about 16 French, about 20 French, about 24 French, or about 26 French. As used herein with reference to the inner diameter D, the term “about” means within plus or minus 1 French of the stated diameter. In certain embodiments, the inner diameter D of the inner liner 232 is the same in each of the regions 122, 124, 126, 128 while, in other embodiments, the inner diameter D can vary along one or more of the regions 122, 124, 126, 128.

In some embodiments, the inner liner 232 or the outer sheath 230 can be omitted. For example, the inner liner 232 can be omitted and the coil layers 234, 236 can be coupled to (e.g., fused to) the outer sheath 230.

FIGS. 3A and 3B are enlarged isometric views of portions of the catheter 120 shown in FIG. 1 in accordance with embodiments of the present technology. Specifically, the portion of the catheter 120 shown in FIG. 3A is a portion of the catheter 120 proximal of the distal terminus 125, and the portion of the catheter shown in FIG. 3B is a portion of the catheter 120 including the distal terminus 125. The outer sheath 230 (FIG. 2) is not shown in FIGS. 3A and 3B for clarity. FIGS. 3C and 3D are side views of the coil layers 234, 236 at, for example, the distal region 126 and the distal tip region 128 shown in FIG. 1 in accordance with embodiments of the present technology. Referring to FIGS. 3A-3D together, the coil layers 234, 236 comprise a plurality of individual wires 340 (e.g., filaments, filars, strands, etc.; individually identified as first wire 340a, a second wire 340b, a third wire 340c, and a fourth wire 340d). For clarity, the individual wires 340a, 340b, 340c, and 340d are shown with different patterns/fills in FIG. 3C, and the coil layers 234, 236 are shown with different patterns/fills in FIG. 3D. Other than the patterns/fills of the wires 340, FIGS. 3C and 3D are identical.

In the illustrated embodiment, the wires 340 extend around the inner liner 232 in a helical or spiral pattern about a longitudinal axis L (FIG. 1) of the catheter 120 in a first direction to form the inner coil layer 234, and the wires 340 double back at a distal terminus 342 (FIGS. 3B-3D) to extend about the inner coil layer 234 in a helical or spiral pattern about the longitudinal axis L in a second direction to form the outer coil layer 236. More specifically, in the illustrated embodiment the wires 340 (i) are wound/coiled about the inner liner 232 in a distal direction D in the inner coil layer 234 such that the helical or spiral pattern of the wires 340 in the inner coil layer 234 has first orientation (e.g., a left-hand orientation) and (ii) are wound/coiled about the inner coil layer 234 in a proximal direction P in the outer coil layer 236 such that the helical or spiral pattern of the wires 340 in the outer coil layer 236 has a second orientation (e.g., a right-hand orientation) opposite to the first orientation.

Referring to FIGS. 3B-D together, the wires 340 each include the distal terminus or distal end portion 342 at which the wire 340 switches directions and transitions from the inner coil layer 234 to the outer coil layer 236. Accordingly, the wires 340 are self-terminating (e.g., continuous) at the distal tip region 128 (FIG. 1) of the catheter 120. In contrast, proximal and distal ends of the wires 340 (not shown) can be individually terminating at the proximal region 122 (FIG. 1) of the catheter 120. In some aspects of the present technology, the wires 340 do not need to be glued, annealed, or otherwise secured together at the distal end portions 342 because the distal end portions 342 are self-terminating—thereby reducing the cost and complexity of manufacturing the catheter 120.

Further, each of the wires 340 includes a first portion (e.g., a first half) wound about the inner liner 232 and forming the inner coil layer 234 and a second portion (e.g., a second half) wound about the inner coil layer 234 and forming the outer coil layer 236. The distal end portion 342 of each of the wires 340 separates the first portion from the second portion. In some embodiments, the coil layers 234, 236 are not secured together such that, for example, the first and second portions of the wires 340 can move relative to one another. Referring to FIGS. 2-3D together, such movement can be limited by the outer sheath 230 and/or the inner liner 232, which can couple/secure the coil layers 234, 236 together. That is, the outer sheath 230 and the inner liner 232 can secure the coil layers 234, 236 in place. In other embodiments, the coil layers 234, 236 are directly secured together via adhesives, welding, etc.

Referring again to FIGS. 3A-3D together, the first wire 340a is positioned between (e.g., adjacent to) the second wire 340b and the fourth wire 340d, the second wire 340b is positioned between the first wire 340a and the third wire 340c, the third wire 340c is positioned between the second wire 340b and the fourth wire 340d, and the fourth wire 340d is positioned between the third wire 340c and the first wire 340a. In the illustrated embodiment the wires 340 are evenly spaced apart from one another by a first pitch P₁ in the inner coil layer 234 and the wires 340 are evenly spaced apart from one another by a second pitch P₂ in the outer coil layer 236. In some embodiments, the first and second pitches P₁₋₂ can be the same along an entire length of the catheter 120. In other embodiments, as described in greater detail below, the first and second pitches P₁₋₂ can be different and/or can vary along the length of the catheter 120 to provide different mechanical characteristics along the length of the catheter 120.

In the illustrated embodiment, the catheter 120 includes four of the wires 340 forming the coil layers 234, 236. In other embodiments, the catheter 120 can include more or fewer of the wires 340 (e.g., 1 wire, 2 wires, 3 wires, 5 wires, 6 wires, 8 wires, 12 wires, or more than 12 wires). The wires 340 can be flat wires (e.g., rolled-flat wires) having a generally rectangular cross-sectional shape with dimensions of between about 0.001-0.005 inch (e.g., about 0.003 inch) by about 0.002-0.025 inch (e.g., about 0.010 inch). In other embodiments, the wires 340 can have other cross-sectional shapes (e.g., circular). The wires can be formed from a metallic or other suitably strong material, such as nickel-titanium alloys (e.g., nitinol), platinum, cobalt-chrome alloys, stainless steel, tungsten, and/or titanium. In some embodiments, some or all of the wires 340 can comprise a shape memory material that is heat-set or otherwise configured to have a predetermined shape. For example, some or all of the wires can be configured to deflect such that the catheter 120 has a pre-shaped portion (e.g., along some or all of the distal region 126 and/or the distal tip region 128) as described in, for example, U.S. patent application Ser. No. 17/529,018, titled “CATHETERS HAVING SHAPED DISTAL PORTIONS, AND ASSOCIATED SYSTEMS AND METHODS,” and filed Nov. 17, 2021, which is incorporated by reference herein in its entirety.

FIGS. 3E-3G are additional side views of the coil layers 234, 236 at, for example, the distal region 126 and the distal tip region 128 shown in FIG. 1 in accordance with embodiments of the present technology. Other than the patterns/fills of the wires 340, FIGS. 3E-3G are identical to FIGS. 3C and 3D. For clarity, FIG. 3E shows the inner coil layer 234 with a single pattern/fill and shows the individual first through fourth wires 340a-d in the outer coil layer 236 with different patterns/fills. That is, the second portions of the wires 340 that form the outer coil layer 236 are shown with different patterns/fills. FIG. 3F shows the outer coil layer 236 with a single pattern/fill and shows the individual first through fourth wires 340a-d in the inner coil layer 234 with different patterns/fills. That is, the first portions of the wires 340 that form the inner coil layer 234 are shown with different patterns/fills. FIG. 3G shows (i) the individual first through fourth wires 340a-d in the inner coil layer 234 with different patterns/fills and (ii) the individual first through fourth wires 340a-d in the outer coil layer 236 with different patterns/fills. That is, the first and second portions of the wires 340 that form the coil layers 234, 236, respectively, are shown with different patterns/fills.

Referring to FIGS. 1-3G together, in some aspects of the present technology the coil layers 234, 236 can be configured to provide the catheter 120 with select characteristics over the entire length of the catheter 120 or in select regions of the catheter 120. Such characteristics include flexibility (e.g., the ability of the catheter 120 to flex/bend laterally away from the longitudinal axis L), pushability (e.g., column strength; e.g., the ability of the catheter 120 to transmit a longitudinal force along the length of the catheter 120 from the proximal region 122 to the distal region 126 and the distal tip region 128), torqueability (e.g., torque response; e.g., the ability of the catheter 120 to transmit a rotational force along the length of the catheter 120 from the proximal region 122 to the distal region 126 and the distal tip region 128), kink resistance (e.g., the ability of the catheter 120 to maintain its cross-sectional profile, such as the inner diameter D, during compressive deformation), hoop strength (e.g., the ability of the catheter 120 to maintain its cross-sectional profile when pressure differentials exist between the lumen 121 of the catheter 120 and the environment surrounding the catheter 120, such as during aspiration of the lumen 121), and/or other characteristics known in the art of catheters. For example, the number of the wires 340 forming the coil layers 234, 236 and/or the pitches P₁₋₂ between the wires 340 in the coil layers 234, 236 can be selected to provide a desired flexibility, pushability, torqueability, kink resistance, hoop strength, etc.

More specifically, for example, increasing the pitches P₁₋₂ between the wires 340 can generally decrease the flexibility of the catheter 120 as the wires 340 extend more longitudinally (e.g., less circularly) about the longitudinal axis L of the catheter 120, while also increasing the pushability of the catheter 120. Conversely, decreasing the pitches P₁₋₂ between the wires 340 can generally increase the flexibility of the catheter 120 as the wires 340 extend more circularly about the longitudinal axis L of the catheter 120, while also decreasing the pushability of the catheter 120. Further, increasing the number of the wires 340 can increase the pushability, torqueability, kink strength, and hoop strength while also decreasing the flexibility of the catheter 120. Conversely, decreasing the number of the wires 340 can decrease the pushability, torqueability, kink strength, and hoop strength while also increasing the flexibility of the catheter 120.

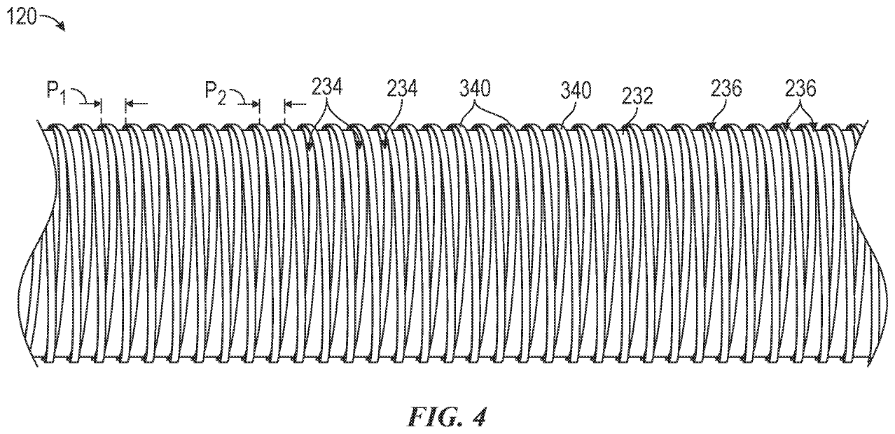

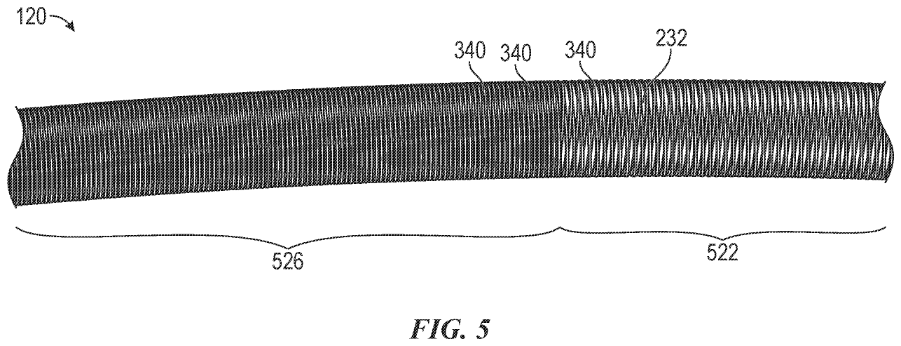

FIGS. 4 and 5, for example, are enlarged isometric views of a portion of the catheter 120 shown in FIG. 1 in accordance with additional embodiments of the present technology. The outer sheath 230 (FIG. 2) is removed in FIGS. 4 and 5 for clarity. Referring first to FIG. 4, in the illustrated embodiment the pitches P₁₋₂ between the wires 340 are smaller (e.g., tighter) than those shown in FIGS. 3A-3G. As described in detail above, the smaller pitches P₁₋₂ can increase the flexibility of the catheter 120. Referring next to FIG. 5, in the illustrated embodiment the pitches P₁₋₂ between the wires 340 in the coil layers 234, 236 are different along different regions of the catheter 120, such as a distal region 526 and a proximal region 522. The pitches P₁₋₂ and the coil layers 234, 236 are not labeled in FIG. 5 for clarity, but are shown in FIGS. 2-4. For example, the pitches P₁₋₂ are smaller in the distal region 526 than in the proximal region 522. Accordingly, as described in detail above, the catheter 120 can be relatively more flexible in the distal region 526 than in the proximal region 522, while the proximal region 522 can provide the catheter 120 with good pushability. More generally, referring to FIGS. 1-5 together, the pitches P₁₋₂ between the wires 340 can vary one or multiple times along the length of the catheter 120 (e.g., in the regions 122, 124, 126, 128) to provide different flexibilities and/or other characteristics along the length of the catheter 120, and/or the pitches P₁₋₂ can vary along the same region of the catheter 120 such that the coil layers 234, 236 impart different flexibilities and/or other characteristics along the same region of the catheter 120.

More generally, in some aspects of the present technology the arrangement of the coil layers 234, 236 can enable the catheter 120 to be steered to and positioned in difficult-to-reach (e.g., tortuous) regions of the anatomy (e.g., venous anatomy) of a patient while still having a relatively large size (e.g., 20 French, 24 French, greater than 24 French). More particularly, the coil layers 234, 236 can give the catheter 120 a torqueability and pushability that is similar to conventional catheters including a braided structure (e.g., a braided mesh) or other reinforcement structure. Similarly, the coil layers 234, 236 can provide a hoop strength and flexibility similar to or greater than conventional catheters including a single-filar coil structure while also reducing kinking around tight bend radii. Further, the coil layers 234, 236 allow the catheter 120 to have these characteristics without including a braided structure or other reinforcement structure, which allows the catheter 120 to be manufactured with a relatively thin inner liner 232 and/or thin outer sheath 230 and results in a catheter 120 having a thin wall. This can enable the inner diameter D of the catheter 120 to be larger than other catheters labeled at the same outer diameter (OD) French size.

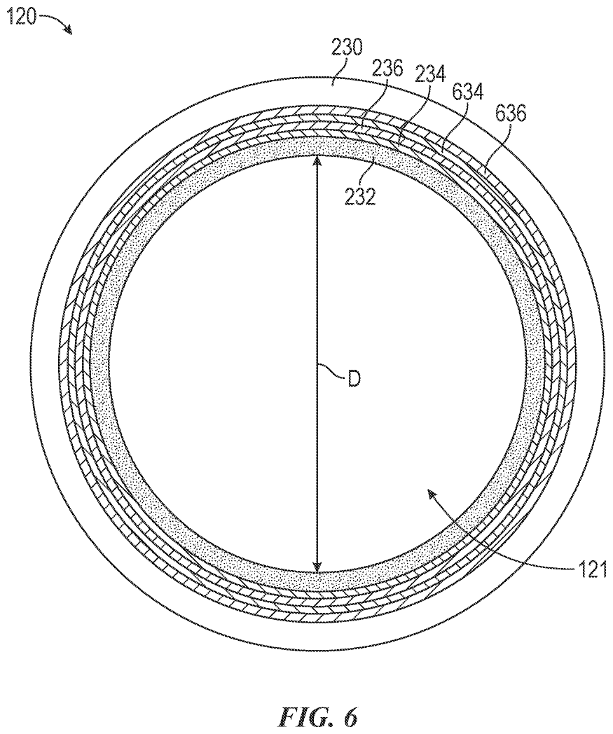

In some embodiments, the catheter 120 can include more coil layers in addition to the coil layers 234, 236. For example, FIG. 6 is a side cross-sectional view of the catheter 120 taken along the line C-C in FIG. 1 in accordance with additional embodiments of the present technology. In the illustrated embodiment, the inner coil layer 234 is a first inner coil layer 234 and the outer coil layer 236 is a first outer coil layer 236 (collectively “first coil layers 234, 236”), and the catheter 120 further includes a second inner coil layer 634 and a second outer coil layer 636 (collectively “second coil layers 634, 636”). The first inner coil layer 234 can be referred to as a first coil layer 234, the first outer coil layer 236 can be referred to as a second coil layer, the second inner coil layer 634 can be referred to as a third coil layer, the second outer coil layer 636 can be referred to as a fourth coil layer, and so on.

The second coil layers 634, 636 can be generally similar or identical to the first coil layers 234, 236. For example, the second coil layers 634, 636 can comprise a plurality of individual wires, and the wires can (i) extend about first outer coil layer 236 in a helical or spiral pattern about the longitudinal axis L (FIG. 1) in a first direction to form the second inner coil layer 634, and (ii) double back to extend about the second inner coil layer 634 in a helical or spiral pattern about the longitudinal axis L in a second direction to form the second outer coil layer 636 over the second inner coil layer 634. Further, the wires that form the second coil layers 634, 636 can be self-terminating at the distal tip region 128 (FIG. 1) of the catheter 120 as described in detail above. The second coil layers 634, 636 can extend along an entire length of the catheter 120 through each of the regions 122, 124, 126, 128 (FIG. 1), or can extend only partially along the length of catheter 120 (e.g., in the distal region 126 and the distal tip region 128). That is, for example, (i) the second coil layers 634, 636 can self-terminate at the same position as the first coil layers 234, 236 such that the catheter 120 includes four coil layers along its entire length (e.g., first coil layers 234, 236 and the second coil layers 634, 636 thereover) or (ii) the second coil layers 634, 636 can self-terminate at a different position from the first coil layers 234, 236 such that a portion of the catheter 120 includes four coil layers (e.g., the first coil layers 234, 236 and the second coil layers 634, 636 thereover) and a portion of the catheter 120 includes two coil layers (e.g., the first coil layers 234, 246). In some embodiments, the wires used to form the first coil layers 234, 236 and the second coil layers 634, 636 can be thinner to maintain the same or similar inner diameter D of the lumen 121 of the catheter 120.

In some embodiments, separate wires are used to form the first coil layers 234, 236 and the second coil layers 634, 636. In other embodiments, the same wires can be wound along the length of the length of the catheter 120 multiple times to form the first coil layers 234, 236 and the second coil layers 634, 636. That is, for example, the same wires can be (i) wound distally along the inner liner 232 to form the first inner coil layer 234, (ii) wound back proximally along the first inner coil layer 234 to form the first outer coil layer 236, (iii) wound back distally along the first outer coil layer 236 to form the second inner coil layer 634, and then (iv) wound back proximally along the second inner coil layer 634 to form the second outer coil layer 636. In such embodiments, the wires can be self-terminating at the distal junction between the first coil layers 234, 236, at the proximal junction between the first outer coil layer 236 and the second inner coil layer 634, and at the distal junction between the second coil layers 634, 636. Further, the number of wires and/or the pitch between the wires in the first coil layers 234, 236 and in second coil layers 634, 636 can vary as described in detail above.

In some aspects of the present technology, the second coil layers 634, 636 can increase the pushability, torqueability, hoop strength, and/or other aspects of the catheter 120. For example, referring to FIGS. 3A-3G and 6 together, torquing the catheter 120 in a first direction (e.g., a clockwise direction) can force the first inner coil layer 234 to compress/close (e.g., acting to decrease the first pitch P₁). Conversely, torquing the catheter 120 in the first direction can simultaneously force the first outer coil layer 236 to expand/open (e.g., acting to increase the second pitch P₂). Likewise, torquing the catheter 120 in a second direction opposite to the first direction (e.g., a counterclockwise direction) can force the first inner coil layer 234 to expand/open while simultaneously forcing the first outer coil layer 236 to compress/close. These opposing forces can create an asymmetrical torque on the catheter 120. The second coil layers 634, 636 can have a similar torque response and, in some aspects of the present technology, can help make the torque response of the catheter 120 more symmetric by balancing the opposing torque response of the first coil layers 234, 236.

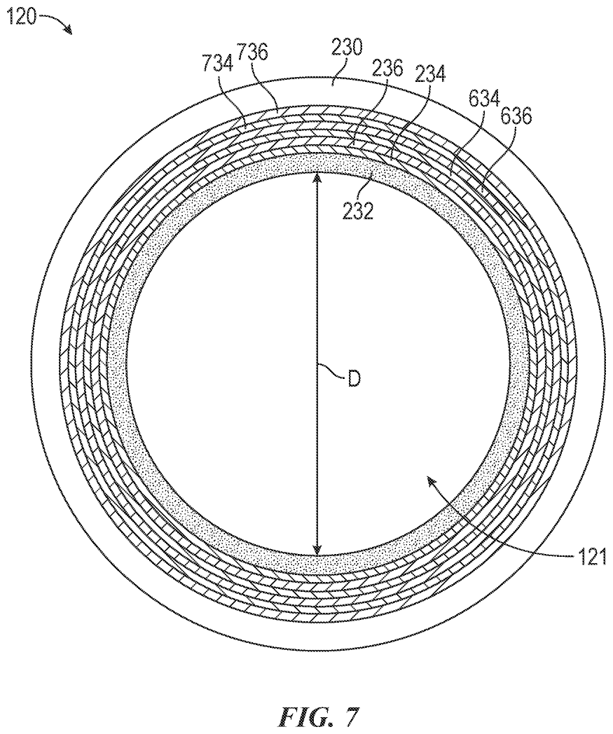

FIG. 7 is a side cross-sectional view of the catheter 120 taken along the line C-C in FIG. 1 in accordance with additional embodiments of the present technology. In the illustrated embodiment, the catheter 120 further includes a third inner coil layer 734 and a third outer coil layer 736 (collectively “the third coil layers 734, 736”). The third coil layers 734, 736 can be generally similar or identical to the first coil layers 234, 236 and the second coil layers 634, 636. For example, the third coil layers 734, 736 can comprise a plurality of individual wires, and the wires can (i) extend about second outer coil layer 636 in a helical or spiral pattern about the longitudinal axis L (FIG. 1) in a first direction to form the third inner coil layer 734, and (ii) double back to extend about the third inner coil layer 734 in a helical or spiral pattern about the longitudinal axis L in a second direction to form the third outer coil layer 736 over the third inner coil layer 734. Further, the wires that form the third coil layers 734, 736 can be self-terminating at the distal tip region 128 (FIG. 1) of the catheter 120 as described in detail above. The third coil layers 734, 736 can extend along an entire length of the catheter 120 through each of the regions 122, 124, 126, 128 (FIG. 1), or can extend only partially along the length of catheter 120 (e.g., in the distal region 126 and the distal tip region 128). In some embodiments, the wires used to form the first coil layers 234, 236, the second coil layers 634, 636, and the third coil layers 734, 736 can be thinner to maintain the same or similar inner diameter D of the lumen 121 of the catheter 120. In other embodiments, the catheter 120 can include more than three coil layers (e.g., four, or more than four coil layers) that can be formed from the same or separate wires.

In some embodiments, separate wires are used to form the first coil layers 234, 236, the second coil layers 634, 636, and the third coil layers 734, 736. In other embodiments, the same wires can be used to form some or all of the coil layers. For example, the same wires can be wound along the length of the catheter 120 multiple times to form the first coil layers 234, 236, the second coil layers 634, 636, and/or the third coil layers 734, 736. That is, for example, the same wires can be (i) wound distally along the inner liner 232 to form the first inner coil layer 234, (ii) wound back proximally along the inner coil layer 234 to form the first outer coil layer 236, (iii) wound back distally along the first outer coil layer 236 to form the second inner coil layer 634, (iv) wound back proximally along the second inner coil layer 634 to form the second outer coil layer 636, (v) wound back distally along the second outer coil layer 636 to form the third inner coil layer 734, and then (vi) wound back proximally along the third inner coil layer 734 to form the third outer coil layer 736. In such embodiments, the wires can be self-terminating at the distal junction between the first coil layers 234, 236, at the proximal junction between the first outer coil layer 236 and the second inner coil layer 634, at the distal junction between the second coil layers 634, 636, at the proximal junction between the second outer coil layer 636 and the third inner coil layer 734, and at the distal junction between the third coil layers 734, 736. Alternatively, for example, the same wires can be used to form the first coil layers 234, 236 and the second coil layers 634, 636 and separate wires can be used to form the third coil layers 734, 736, or the same wires can be used to form the second coil layers 634, 636 and the third coil layers 734, 736 and separate wires can be used to form the first coil layers 234, 236, etc. Further, the number of wires and/or the pitch between the wires in the first coil layers 234, 236, the second coil layers 634, 636, and the third coil layers 734, 736 can vary as described in detail above.

In some aspects of the present technology, the third coil layers 734, 736 can further increase the pushability, torqueability, hoop strength, and/or other characteristics of the catheter 120. For example, the third coil layers 734, 736 can help make the torque response of the catheter 120 more symmetric by balancing the opposing torque response of the first coil layers 234, 236 and/or the second coil layers 634, 636.

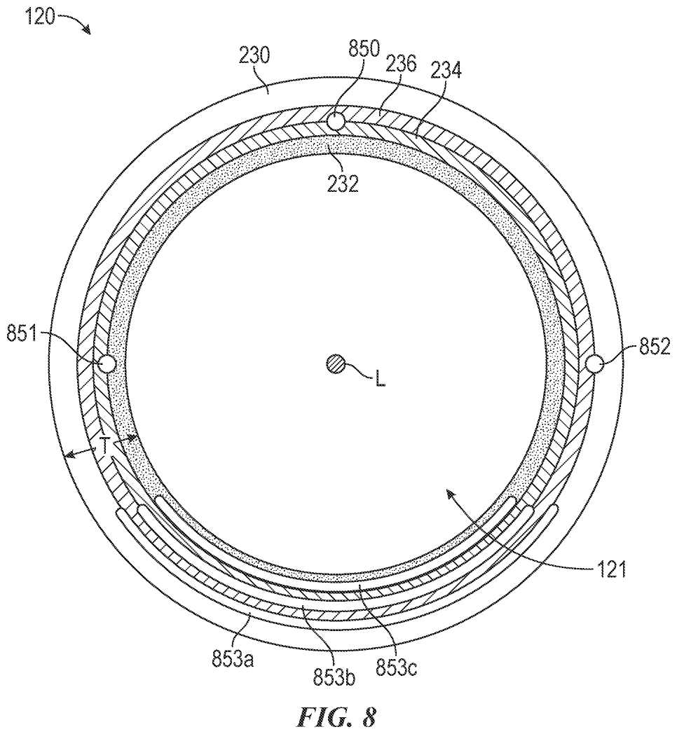

In some embodiments, the catheter 120 can include one or more lumens, pull wires, and/or other components. For example, FIG. 8 is a side cross-sectional view of the catheter 120 taken along the line C-C in FIG. 1 in accordance with additional embodiments of the present technology. In the illustrated embodiment, the catheter 120 includes a first component 850 positioned between the coil layers 234, 236, a second component 851 positioned between the inner liner 232 and the inner coil layer 234, and a third component 852 positioned between the outer sheath 230 and the outer coil layer 236. The catheter 120 can include any number of the components 850-852 positioned at different circumferential positions about the lumen 121.

One or more of the first through third components 850, 851, 852 (“components 850, 851, 852”) can be lumens through which other devices can be inserted and/or through which fluid can be injected or withdrawn (e.g., to inflate a balloon coupled to the catheter 120). In some aspects of the present technology, where one or more of the components 850, 851, 852 comprises an inflation lumen, the coil layers 234, 236 can provide support for the inflation lumen while also allowing for improved (e.g., minimized) inflation/deflation times even when the catheter 120 is positioned in highly tortuous anatomy. In some embodiments, one or more of the components 850, 851, 852 comprise a lumen through which contrast fluid can be injected to facilitate visualization of a distal portion of the catheter 120 (e.g., the distal region 126 and/or the distal tip region 128 shown in FIG. 1). In such embodiments, the components 850, 851, 852 can comprise tubes, extrusions, elongate members, and/or the like. The lumens can have a circular cross-sectional shape as illustrated in FIG. 8, or can have other cross-sectional shapes. For example, first through third lumens 853a-c (e.g., contrast injection lumens), respectively, are illustrated in FIG. 8 as having a U-like cross sectional shape. More specifically, the lumens 853a-c can extend partially or fully circumferentially about the longitudinal axis L of the catheter 120. In the illustrated embodiment, the first lumen 853a extends between the outer sheath 850 and the outer coil layer 856, the second lumen 853b extends between the coil layers 854, 856, and the third lumen 853c extends between the inner coil layer 854 and the inner liner 852.

In some embodiments, one or more of the components 850, 851, 852 can comprise a pull wire configured to, for example, deflect a portion of the catheter 120 (e.g., the distal region 126 and/or the distal tip region 128 shown in FIG. 1). For example, the pull wire can be secured at a distal end to one or both of the coil layers 234, 236 and can be proximally withdrawn to deflect the catheter 120. In some embodiments, where the catheter 120 includes more than the two illustrated coil layers 234, 236 (e.g., as described in detail above with reference to FIGS. 6 and 7), one or more of the components 850, 851, 852 can be positioned between different ones (e.g., adjacent ones) of the coil layers. For example, multiple inflation and/or fluid injection lumens can be positioned between different coil layers to facilitate inflation of multiple balloons.

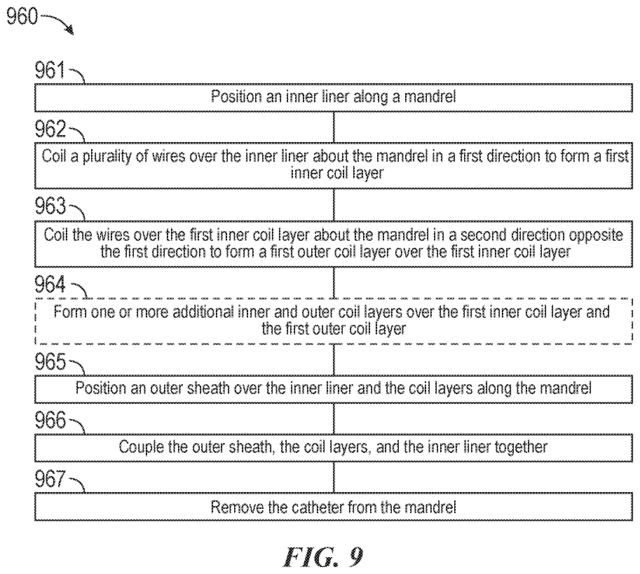

In some aspects of the present technology, the first component 850 can be secured in position between the coil layers 234, 236 during manufacturing by (i) positioning the first component over the inner coil layer 234 after winding the wires 340 (FIGS. 3A-3G) about the inner liner 232 and then (ii) further winding the wires 340 over the first component 850 and the inner coil layer 234 to form the outer coil layer 236. In some aspects of the present technology, as described in further detail below with reference to FIG. 9, the catheter 120 can be manufactured to include the components 850, 851, 852 with a single machine setup due to the self-terminating ends of the coil layers 234, 236. In additional aspects of the present technology, the components 850, 851, 852 can be formed within the catheter 120 without increasing or substantially increasing a thickness T of the catheter 120 compared to conventional manufacturing methods.

### III. SELECTED EMBODIMENTS OF METHODS OF MANUFACTURING CATHETERS HAVING SELF-TERMINATING COILS

FIG. 9 is a flow diagram of a process or method 960 for manufacturing a catheter (e.g., the catheter 120) in accordance with embodiments of the present technology. Although some features of the method 960 are described in the context of the catheter 120 shown in FIGS. 1-8 for the sake of illustration, one skilled in the art will readily understand that the method 960 can be carried out to form other catheters described herein.

At block 961, the method 960 can include positioning the inner liner 232 along a mandrel, hyoptube, or another elongate member. In some embodiments, the inner liner 232 is stretched along the mandrel to have a desired thickness.

At block 962, the method 960 can include coiling/winding the wires 340 over the inner liner 232 about the mandrel in a first direction (e.g., the distal direction D) to form the inner coil layer 234. As described in detail above, the number of the wires 340 and/or the pitch P₁ between the wires 340 can be varied to provide desired characteristics of the catheter 120. In some embodiments, tension is applied to the wires 340 as they are coiled over the inner liner 232 such that the wires 340 tightly wrap around the inner liner 232. The wires 340 can be coiled manually by a user or by a machine.

At block 963, the method 960 can include coiling/winding the wires 340 over the inner coil layer 234 about the mandrel in a second direction (e.g., the proximal direction P) opposite the first direction to form the outer coil layer 236. That is, the direction of coiling can be reversed to form the distal end portions 342 of the wires 340 at, for example, the distal tip region 128 of the catheter 120. As described in detail above, the pitch P₂ between the wires 340 can be varied to provide desired characteristics of the catheter 120. In some embodiments, tension is applied to the wires 340 as they are coiled over the inner coil layer 234 such that the wires 340 tightly wrap around the inner coil layer 234.

At block 964, the method 960 can optionally include forming one or more additional inner and outer coil layers over the coil layers 234, 236, such as the second coil layers 634, 636, the third coil layers 734, 736, and/or additional coil layers. As described in detail above, the additional inner and outer coil layers can be formed in the same manner as the coil layers 234, 236 (blocks 962 and 963), and can be formed using separate wires and/or the same ones of the wires 340 used to form the coil layers 234, 236.

At block 965, the method 960 can include positioning the outer sheath 230 over the inner liner 232 and the coil layers 234, 236 (and any additional coil layers). In some embodiments, the outer sheath 230 is stretched along the mandrel to have a desired thickness.

At block 966, the method 960 can include coupling the outer sheath 230, the coil layers 234, 236 (and any additional coil layers), and the inner liner 232 together to form the catheter 120. For example, the outer sheath 230 and the inner liner 232 can be heat shrunk, fused, laminated, or otherwise secured together with the coil layers 234, 236 therebetween.

Finally, at block 967, the method 960 can include removing the catheter 120 from the mandrel. In some embodiments, the method 960 can optionally include positioning one or more components (e.g., the components 850-852), such as a lumen or pull wire, between any of the steps described in blocks 961-965 such that the components are integrally formed in the wall of the catheter 120.

### IV. SELECTED EMBODIMENTS OF METHODS OF CLOT TREATMENT

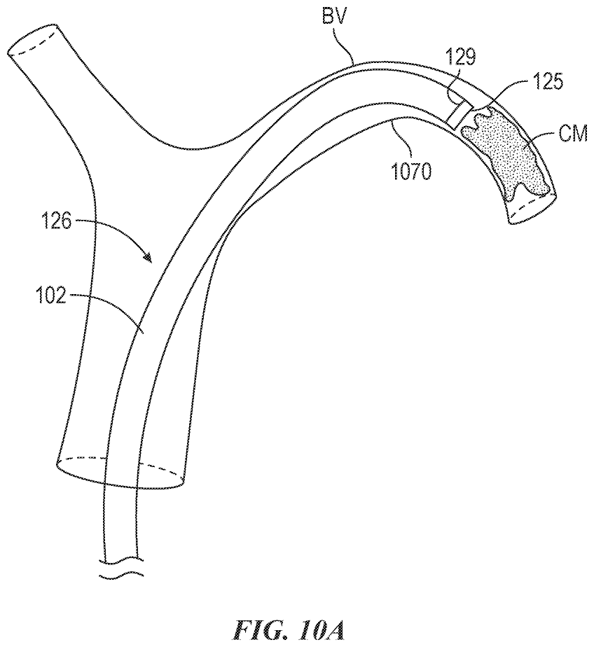

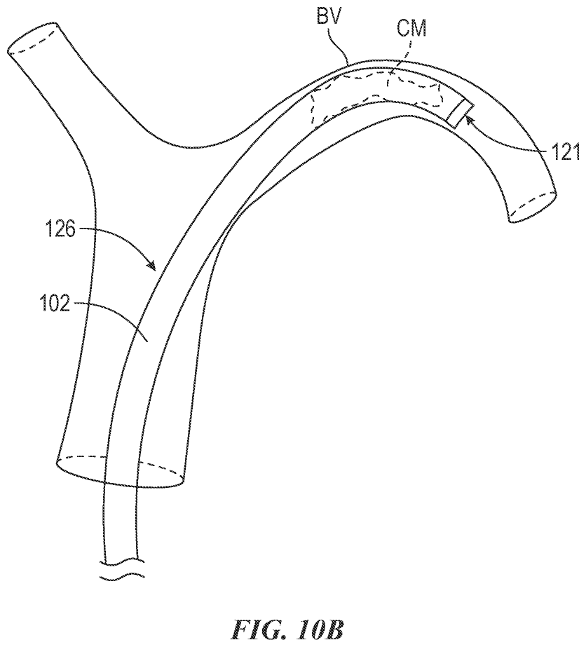

FIGS. 10A and 10B are side views of the distal region 126 of the catheter 120 of the clot treatment system 100 during a procedure for removing clot material CM (e.g., a pulmonary embolism) from within a blood vessel BV (e.g., a pulmonary blood vessel) of a patient (e.g., a human patient) in accordance with embodiments of the present technology. As noted above, in some embodiments the clot removal procedure illustrated in FIGS. 10A and 10B can be generally similar or identical to any of the clot removal procedures disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

With reference to FIGS. 1 and 10A together, the catheter 120 can be advanced through the patient to proximate the clot material CM with the blood vessel BV (e.g., advanced to a treatment site within the blood vessel BV). In some embodiments, the catheter 120 is advanced through the blood vessel BV until the distal terminus 125 of the catheter 120 is positioned proximate to a proximal portion of the clot material CM. The position of the distal terminus 125 can be confirmed or located via visualization of the marker band 129 using fluoroscopy or another imaging procedure (e.g., a radiographic procedure). In other embodiments, the distal terminus 125 can be positioned at least partially within the clot material CM or distal of the clot material CM.

In some aspects of the present technology, the catheter 120 is configured to flex/bend into tortuous (e.g., hard-to-reach) regions of the blood vessel BV. For example, in the illustrated embodiment the catheter 120 has flexed around a bend 1070 in the blood vessel BV that can have a relatively small radius of curvature. The portion of the blood vessel BV distal of the bend 1070 can be difficult to reach due to the required approach angle, varying anatomy of the blood vessel BV, and/or irregularities due to illness of the patient. In some embodiments, the blood vessel BV can be a portion of left pulmonary artery, the temporal arteries, the inferior vena cava, or the right atrium. In some embodiments, the clot material CM can be a clot in transit (CIT) within the right atrium.

Access to the pulmonary vessels can be achieved through the patient's vasculature, for example, via the femoral vein. In some embodiments, the clot treatment system 100 can include an introducer (e.g., a Y-connector with a hemostasis valve; not shown) that can be partially inserted into the femoral vein. A guidewire (not shown) can be guided into the femoral vein through the introducer and navigated through the right atrium, the tricuspid valve, the right ventricle, the pulmonary valve, and into the main pulmonary artery. Depending on the location of the clot material CM, the guidewire can be guided to one or more of the branches of the right pulmonary artery and/or the left pulmonary artery. In some embodiments, the guidewire can be extended entirely or partially through the clot material CM. In other embodiments, the guidewire can be extended to a location just proximal of the clot material CM. After positioning the guidewire, the catheter 120 can be placed over the guidewire and advanced to the position proximate to the clot material CM as illustrated in FIG. 10A. In some embodiments, the guidewire can then be withdrawn while, in other embodiments, the guidewire can remain and can be used to guide other catheters (e.g., delivery catheters, additional aspiration guide catheters), interventional devices, etc., to the treatment site. It will be understood, however, that other access locations into the venous circulatory system of a patient are possible and consistent with the present technology. For example, the user can gain access through the jugular vein, the subclavian vein, the brachial vein, or any other vein that connects or eventually leads to the superior vena cava. Use of other vessels that are closer to the right atrium of the patient's heart can also be advantageous as it reduces the length of the instruments needed to reach the clot material CM.

With reference to FIGS. 1 and 10B together, the pressure source 106 is configured to generate (e.g., form, create, charge, build-up) a vacuum (e.g., negative relative pressure) and store the vacuum for subsequent application to the catheter 120. For example, after positioning the catheter 120 proximate the clot material CM, a user can first close the fluid control device 114 before generating the vacuum in the pressure source 106 by, for example, withdrawing the plunger of a syringe coupled to the connector 116. In this manner, a vacuum is charged within the pressure source 106 (e.g., a negative pressure is maintained) before the pressure source 106 is fluidly connected to the lumen 121 of the catheter 120. To aspirate the lumen 121 of the catheter 120, the user can open the fluid control device 114 to fluidly connect the pressure source 106 to the catheter 120 and thereby apply or release the vacuum stored in the pressure source 106 to the lumen 121 of the catheter 120.

Opening of the fluid control device 114 instantaneously or nearly instantaneously applies the stored vacuum pressure to the tubing assembly 110 and the catheter 120, thereby generating a suction pulse throughout the catheter 120. In particular, the suction is applied at the distal tip region 128 of the catheter 120 to suck/aspirate at least a portion of the clot material CM into the lumen 121 of the catheter 120, as shown in FIG. 10B. In one aspect of the present technology, pre-charging or storing the vacuum in the pressure source 106 before applying the vacuum to the lumen 121 of the catheter 120 is expected to generate greater suction forces and corresponding fluid flow velocities at and/or near the distal tip region 128 of the catheter 120 compared to simply activating the pressure source 106 while it is fluidly connected to the catheter 120. In other embodiments, the pressure source 106 can be activated while the fluid control device 114 is open to aspirate the clot material CM.

Sometimes, as shown in FIG. 10B, discharging the vacuum stored in the pressure source to aspirate the lumen 121 of the catheter 120 may remove substantially all (e.g., a desired amount) of the clot material CM from the blood vessel BV. That is, a single aspiration pulse may adequately remove the clot material CM from the blood vessel BV. In other embodiments, a portion of the clot material CM may remain in the blood vessel BV. In such instances, the user may wish to again apply vacuum pressure (conduct an “aspiration pass”) to remove all or a portion of the remaining clot material CM in the blood vessel BV. In such instances, the pressure source 106 can be disconnected from the tubing assembly 110 and drained (e.g., aspirated clot removal removed) before the pressure source 106 is reconnected to the tubing assembly 110 and activated once again. After removing a desired amount of the clot material CM, the catheter 120 can be withdrawn from the patient.

In some aspects of the present technology, the relatively great flexibility and torqueability of the catheter 120 (e.g., as provided by the coil layers 234, 236 shown in FIGS. 3A-3G) allow the catheter 120 to be positioned in difficult-to-reach areas of the blood vessel BV (or elsewhere in the vasculature of the patient) without reducing the size of the lumen 121. It is expected that the increased size of the lumen 121 will provide greater suction forces over a smaller period of time (e.g., will provide a larger vacuum impulse). In some embodiments, the greater suction forces can facilitate the removal of clot material from a blood vessel of a patient even where the clot material is strongly lodged or attached within the blood vessel (e.g., a chronic clot). Accordingly, in contrast to conventional catheters, the catheter 120 can be used to generate greater aspirational forces for improved clot removal in hard-to-reach places of the vasculature. In additional aspects of the present technology, the coil layers 234, 236 (FIGS. 3A-3G) can provide a high hoop strength along the distal region 126 of the catheter 120 that inhibits or even prevents kinking or other unwanted movement of the catheter 120 when the pressure source 106 is used to generate a suction pulse at the distal region 126 of the catheter 120.

### V. ADDITIONAL EXAMPLES

Several aspects of the present technology are set forth in the following examples:

1. A catheter, comprising:
a longitudinal axis;
a plurality of wires, wherein the wires are coiled about the longitudinal axis spaced apart from another in a first direction to form a first coil layer, and wherein the wires are coiled about the longitudinal axis spaced apart from another in a second direction, opposite the first direction, over the first coil layer to form a second coil layer over the first coil layer; and
an outer sheath over the plurality of wires.

2. The catheter of example 1, further comprising an inner liner, wherein the wires are coiled over the inner liner.

3. The catheter of example 1 or example 2 wherein the wires in the first coil layer have a helical arrangement having a first orientation, and wherein the wires in the second coil layer have a helical arrangement having a second orientation opposite the first orientation.

4. The catheter of any one of examples 1-3 wherein the wires each include a continuous end portion at which the wire switches from the first direction to the second direction and transitions from the first coil layer to the second coil layer.

5. The catheter of any one of examples 1˜4 wherein the wires each include a self-terminating end portion at which the wire switches from the first direction to the second direction and transitions from the first coil layer to the second coil layer.

6. The catheter of any one of examples 1-5 wherein the wires have a first pitch in the first coil layer and a second pitch in the second coil layer, and wherein the first pitch and the second pitch are the same.

7. The catheter of any one of examples 1-6 wherein the wires have a first pitch in the first coil layer and a second pitch in the second coil layer, and wherein the first pitch and the second pitch are the different.

8. The catheter of any one of examples 1-7 wherein the wires have a first pitch in the first coil layer and a second pitch in the second coil layer, and wherein the first pitch and/or the second pitch varies along the longitudinal axis.

9. The catheter of any one of examples 1-8 wherein the wires are further (a) coiled about the longitudinal axis in the first direction to form a third coil layer over the second coil layer and (b) coiled about the longitudinal axis in the second direction over the third coil layer to form a fourth coil layer over the third coil layer.

10. The catheter example 9 wherein the wires each include (a) a first self-terminating end portion at which the wire switches from the first direction to the second direction and transitions from the first coil layer to the second coil layer and (b) a second self-terminating end portion at which the wire switches from the second direction to the first direction and transitions from the second coil layer to the third coil layer.

11. The catheter of example 10 wherein the first self-terminating end portions are positioned at a distal end portion of the catheter, and wherein the second self-terminating end portions are positioned at a proximal end portion of the catheter.

12. The catheter of example 10 or example 11 wherein the first self-terminating end portions are at a different position along the longitudinal axis relative to the second self-terminating end portions.

13. The catheter of any of examples 9-12 wherein the third coil layer extends only partially over the second coil layer along the longitudinal axis.

14. The catheter of any of examples 9-12 wherein the third coil layer extends entirely over the second coil layer along the longitudinal axis.

15. The catheter of any one of examples 1-8 wherein the wires are first wires, and further comprising a plurality of second wires, wherein the second wires are (a) coiled about the longitudinal axis in the first direction to form a third coil layer over the second coil layer and (b) coiled about the longitudinal axis in the second direction over the third coil layer to form a fourth coil layer over the third coil layer.

16. The catheter example 15 wherein the first wires each include a first self-terminating end portion at which the first wire switches from the first direction to the second direction and transitions from the first coil layer to the second coil layer, and wherein the second wires each include a second self-terminating end portion at which the second wire switches from the first direction to the second direction and transitions from the third coil layer to the fourth coil layer.

17. The catheter of example 16 wherein the first self-terminating end portions are positioned at a same position along the longitudinal axis relative to the second self-terminating end portions.

18. The catheter of example 17 wherein the first self-terminating end portions and the second self-terminating end portions are positioned at a distal end portion of the catheter.

19. The catheter of example 16 wherein the first self-terminating end portions are positioned at a different position along the longitudinal axis relative to the second self-terminating end portions.

20. The catheter of any of examples 15-19 wherein the third coil layer extends only partially over the second coil layer along the longitudinal axis.

21. The catheter of any of examples 15-19 wherein the third coil layer extends entirely over the second coil layer along the longitudinal axis.

22. The catheter of any one of examples 1-21 wherein the wires have a rectangular cross-sectional shape.

23. The catheter of any one of examples 1-22 wherein the plurality of wires comprises 4 wires.

24. The catheter of any one of examples 1-22 wherein the plurality of wires comprises 12 wires.

25. The catheter of any one of examples 1-24, further comprising an inner liner defining a lumen, wherein the wires are coiled over the inner liner, wherein the lumen has a diameter of 20 French or greater.

26. The catheter of any one of examples 1-25, further comprising a lumen extending between the inner coil layer and the outer coil layer.

27. The catheter of any one of examples 1-26, further comprising a lumen extending between the outer coil layer and the outer sheath.

28. The catheter of any one of examples 1-27, further comprising:
an inner liner, wherein the wires are coiled over the inner liner; and
a lumen extending between the inner coil layer and the inner liner.

29. A method of manufacturing a catheter, the method comprising:
coiling a plurality of wires about a mandrel in a first direction to form an inner coil layer;
coiling the wires about the mandrel in a second direction opposite the first direction to form an outer coil layer over the inner coil layer;
positioning an outer sheath over the inner and outer coil layers; and
coupling the outer sheath to the inner and outer coil layers.

30. The method of example 29 wherein the method further comprises positioning an inner liner over the mandrel, and wherein coiling the wires about the mandrel in the first direction includes coiling the wires over the inner liner in the first direction to form the inner coil layer over the inner liner.

31. The method of example 30 wherein coupling the outer sheath to the inner and outer coil layers includes fusing the outer sheath and the inner liner together.

32. The method of any one of examples 29-31 wherein the wires in the first coil layer have a helical arrangement having a first orientation, and wherein the wires in the second coil layer have a helical arrangement having a second orientation opposite the first orientation.

33. The method of any one of examples 29-32 wherein coiling the wires about the mandrel in the first direction to form the inner coil layer includes varying a pitch between the wires in the first direction.

34. The method of any one of examples 29-33 wherein coiling the wires about the mandrel in the second direction to form the outer coil layer includes varying a pitch between the wires in the second direction.

35. The method of any one of examples 29-34 wherein the method further comprises positioning an elongate component over the inner coil layer, and wherein coiling the wires about the mandrel in the second direction to form the outer coil layer includes coiling the wires over the elongate component to secure the elongate component between the inner and outer coil layers.

36. The method of example 35 wherein the elongate component comprises a lumen.

37. The method of any one of examples 29-36 wherein coiling the wires about the mandrel in the second direction includes forming a continuous end portion of each of the wires at which the wire switches from the first direction to the second direction and transitions from the inner coil layer to the outer coil layer.

38. The method of any one of examples 29-37 wherein coiling the wires about the mandrel in the second direction includes forming a self-terminating end portion of each of the wires at which the wire switches from the first

39. A catheter, comprising:
a longitudinal axis;
a plurality of wires, wherein—
   the wires are coiled about the longitudinal axis in a first direction to form a first coil layer,
   the wires are coiled about the longitudinal axis in a second direction, opposite the first direction, over the first coil layer to form a second coil layer over the first coil layer, and
   the wires each include a continuous end portion at which the wire switches from the first direction to the second direction and transitions from the inner coil layer to the outer coil layer; and
an outer sheath over the plurality of wires.

40. A catheter, comprising:
a longitudinal axis;
a plurality of wires, wherein—
   the wires are coiled about the longitudinal axis in a first direction to form a first coil layer,
   the wires are coiled about the longitudinal axis in a second direction, opposite the first direction, over the first coil layer to form a second coil layer over the first coil layer, and
   the wires each include a self-terminating end portion at which the wire switches from the first direction to the second direction and transitions from the first coil layer to the second coil layer; and
an outer sheath over the wires.

### VI. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A catheter, comprising:
a longitudinal axis;
a plurality of wires, wherein the wires are coiled about the longitudinal axis spaced apart from another in a first direction to form a first coil layer, and wherein the wires are coiled about the longitudinal axis spaced apart from one another in a second direction, opposite the first direction, over the first coil layer to form a second coil layer over the first coil layer; and
an outer sheath over the plurality of wires.

2. The catheter of claim 1, further comprising an inner liner, wherein the wires are coiled over the inner liner.

3. The catheter of claim 1 wherein the wires in the first coil layer have a helical arrangement having a first orientation, and wherein the wires in the second coil layer have a helical arrangement having a second orientation opposite the first orientation.

4. The catheter of claim 1 wherein the wires each include a continuous end portion at which the wire switches from the first direction to the second direction and transitions from the first coil layer to the second coil layer.

5. The catheter of claim 1 wherein the wires each include a self-terminating end portion at which the wire switches from the first direction to the second direction and transitions from the first coil layer to the second coil layer.

6. The catheter of claim 1 wherein the wires have a first pitch in the first coil layer and a second pitch in the second coil layer, and wherein the first pitch and the second pitch are the same.

7. The catheter of claim 1 wherein the wires have a first pitch in the first coil layer and a second pitch in the second coil layer, and wherein the first pitch and the second pitch are the different.

8. The catheter of claim 1 wherein the wires have a first pitch in the first coil layer and a second pitch in the second coil layer, and wherein the first pitch and/or the second pitch varies along the longitudinal axis.

9. The catheter of claim 1 wherein the wires are further (a) coiled about the longitudinal axis in the first direction to form a third coil layer over the second coil layer and (b) coiled about the longitudinal axis in the second direction over the third coil layer to form a fourth coil layer over the third coil layer.

10. The catheter of claim 9 wherein the wires each include (a) a first self-terminating end portion at which the wire switches from the first direction to the second direction and transitions from the first coil layer to the second coil layer and (b) a second self-terminating end portion at which the wire switches from the second direction to the first direction and transitions from the second coil layer to the third coil layer.

11. The catheter of claim 10 wherein the first self-terminating end portions are positioned at a distal end portion of the catheter, and wherein the second self-terminating end portions are positioned at a proximal end portion of the catheter.

12. The catheter of claim 10 wherein the first self-terminating end portions are at a different position along the longitudinal axis relative to the second self-terminating end portions.

13. The catheter of claim 9 wherein the third coil layer extends only partially over the second coil layer along the longitudinal axis.

14. The catheter of claim 9 wherein the third coil layer extends entirely over the second coil layer along the longitudinal axis.

15. The catheter of claim 1 wherein the wires are first wires, and further comprising a plurality of second wires, wherein the second wires are (a) coiled about the longitudinal axis in the first direction to form a third coil layer over the second coil layer and (b) coiled about the longitudinal axis in the second direction over the third coil layer to form a fourth coil layer over the third coil layer.

16. The catheter of claim 15 wherein the first wires each include a first self-terminating end portion at which the first wire switches from the first direction to the second direction and transitions from the first coil layer to the second coil layer, and wherein the second wires each include a second self-terminating end portion at which the second wire switches from the first direction to the second direction and transitions from the third coil layer to the fourth coil layer.

17. The catheter of claim 16 wherein the first self-terminating end portions are positioned at a same position along the longitudinal axis relative to the second self-terminating end portions.

18. The catheter of claim 16 wherein the first self-terminating end portions and the second self-terminating end portions are positioned at a distal end portion of the catheter.

19. The catheter of claim 16 wherein the first self-terminating end portions are positioned at a different position along the longitudinal axis relative to the second self-terminating end portions.

20. The catheter of claim 15 wherein the third coil layer extends only partially over the second coil layer along the longitudinal axis.

21. The catheter of claim 15 wherein the third coil layer extends entirely over the second coil layer along the longitudinal axis.

22. The catheter of claim 1 wherein the wires have a rectangular cross-sectional shape.

23. The catheter of claim 1 wherein the plurality of wires comprises 4 wires.

24. The catheter of claim 1 wherein the plurality of wires comprises 12 wires.

25. The catheter of claim 1, further comprising an inner liner defining a lumen, wherein the wires are coiled over the inner liner, wherein the lumen has a diameter of 20 French or greater.

26. The catheter of claim 1, further comprising a lumen extending between the inner coil layer and the outer coil layer.

27. The catheter of claim 1, further comprising a lumen extending between the outer coil layer and the outer sheath.

28. The catheter of claim 1, further comprising:
an inner liner, wherein the wires are coiled over the inner liner; and
a lumen extending between the inner coil layer and the inner liner.

29. A method of manufacturing a catheter, the method comprising:
coiling a plurality of wires about a mandrel in a first direction to form an inner coil layer;
coiling the wires about the mandrel in a second direction opposite the first direction to form an outer coil layer over the inner coil layer;
positioning an outer sheath over the inner and outer coil layers; and
coupling the outer sheath to the inner and outer coil layers.

30. The method of claim 29 wherein the method further comprises positioning an inner liner over the mandrel, and wherein coiling the wires about the mandrel in the first direction includes coiling the wires over the inner liner in the first direction to form the inner coil layer over the inner liner.

31. The method of claim 30 wherein coupling the outer sheath to the inner and outer coil layers includes fusing the outer sheath and the inner liner together.

32. The method of claim 29 wherein the wires in the first coil layer have a helical arrangement having a first orientation, and wherein the wires in the second coil layer have a helical arrangement having a second orientation opposite the first orientation.

33. The method of claim 29 wherein coiling the wires about the mandrel in the first direction to form the inner coil layer includes varying a pitch between the wires in the first direction.

34. The method of claim 29 wherein coiling the wires about the mandrel in the second direction to form the outer coil layer includes varying a pitch between the wires in the second direction.

35. The method of claim 29 wherein the method further comprises positioning an elongate component over the inner coil layer, and wherein coiling the wires about the mandrel in the second direction to form the outer coil layer includes coiling the wires over the elongate component to secure the elongate component between the inner and outer coil layers.

36. The method of claim 35 wherein the elongate component comprises a lumen.

37. The method of claim 29 wherein coiling the wires about the mandrel in the second direction includes forming a continuous end portion of each of the wires at which the wire switches from the first direction to the second direction and transitions from the inner coil layer to the outer coil layer.

38. The method of claim 29 wherein coiling the wires about the mandrel in the second direction includes forming a self-terminating end portion of each of the wires at which the wire switches from the first direction to the second direction and transitions from the inner coil layer to the outer coil layer.

39. A catheter, comprising:
a longitudinal axis;
a plurality of wires, wherein—
the wires are coiled about the longitudinal axis in a first direction to form a first coil layer,
the wires are coiled about the longitudinal axis in a second direction, opposite the first direction, over the first coil layer to form a second coil layer over the first coil layer, and
the wires each include a self-terminating end portion at which the wire switches from the first direction to the second direction and transitions from the first coil layer to the second coil layer; and
an outer sheath over the wires.
